SEQUENCE LISTING (1 sequence)

SEQ ID NO: 1 - protein, 8 aa, Artificial Sequence
  Synthetic construct
FCFWKTCT

(19) United States
(12) Patent Application Publication
(10) Pub. No.: US 2013/0224300 A1
(43) Pub. Date: Aug. 29, 2013

(54) COMPOSITIONS AND METHODS THEREOF FOR ORAL ADMINISTRATION OF DRUGS

(71) Applicant: Edward T. Maggio, San Diego, CA (US)

(72) Inventor: Edward T. Maggio, San Diego, CA (US)

(21) Appl. No.: 13/594,478

(22) Filed: Aug. 24, 2012

Related U.S. Application Data
(60) Provisional application No. 61/527,788, filed on Aug. 26, 2011.

(51) Int. Cl. A61K 47/22; A61K 47/10

(52) U.S. Cl. CPC A61K 47/22; A61K 47/10
USPC 424/484; 514/777; 514/774

(57) ABSTRACT

The present invention provides therapeutic compositions including a therapeutic agent in a non-aqueous matrix having an absorption enhancer and therapeutic agent, as well as methods for administering such compositions and providing enhanced oral bioavailability.

### CROSS REFERENCE TO RELATED APPLICATION(S)

This application claims the benefit of priority under 35 U.S.C. §119(e) of U.S. Ser. No. 61/527,788, filed Aug. 26, 2011, the entire contents of which is incorporated herein by reference in its entirety.

### BACKGROUND OF THE INVENTION

1. Field of the Invention

This invention relates generally to therapeutic compositions, and more particularly to compositions including a therapeutic agent in a non-aqueous matrix including an absorption enhancer, as well as methods for administering such compositions and providing enhanced oral bioavailability.

2. Background Information

The FDA classifies drug substances into four categories under the Biopharmaceutical Classification System (BCS). Class I drugs exhibit high permeability and high water solubility. Class II drugs exhibit high permeability and low water solubility. Class III drugs exhibit low permeability and high water solubility. Class IV drugs exhibit low permeability and low water solubility. In general, the more hydrophobic or lipophilic a molecule is the poorer its solubility in water and conversely the higher its solubility in a non-aqueous matrix or solvent.

It is estimated that that up to 40% of new chemical entities (NCE's) discovered by the pharmaceutical industry today are poorly water-soluble. Solubility issues also complicate the delivery of many existing drugs. The ability to deliver poorly water-soluble drugs will grow in significance in coming years as NCE's are relied upon for a larger share of the pharmaceutical market by innovator companies. Similarly, generic drug manufacturers need to employ economically efficient methods to deliver drugs with poor water solubility as such drugs go off patent. Relative to highly water-soluble compounds, low drug solubility in water manifests itself in a number of in vivo consequences, including decreased bioavailability, increased chance of food effect, and higher inter-patient and interdose variability. New formulations and methods that facilitate administration of drugs with at least some solubility in non-aqueous matrices are needed.

A number of technologies have been developed to improve solubility enhancement. These include particle size reduction. By reducing particle size, the increased surface area may improve the dissolution properties of a drug in a wider range of formulation approaches and delivery technologies. Conventional methods of particle size reduction include spray drying, micronization, milling, and grinding. These mechanical methods often impart significant amounts of physical and thermal stress on the drug product which may induce varying degrees of degradation. Particle size reduction methods, such as grinding and milling are often incapable of reducing particle size of nearly insoluble of nearly water insoluble drugs. Poorly water-soluble drugs are most often soluble in non-aqueous solvents.

Small molecule organic drugs exhibit a range of water solubilities, with some being highly soluble and some being very poorly soluble. Still other drugs are amphiphilic being soluble in both water and hydrophobic solvents. Many peptides are amphiphilic owing to the hydrophobic and hydrophilic properties of the amino acyl side chains. Similarly, in spite of the many attractive aspects of peptides as potential therapeutic agents, many peptides, whether linear or cyclic, monomeric, or multi-chained, are poorly soluble in water.

Absorption enhancer molecules have been formulated into aqueous solution for administration to the nasal mucosa. Such aqueous solutions have in many cases been effective in delivering peptides and proteins into systemic circulation. When mixtures of these absorption enhancers and peptides or non-peptide drugs are administered into the nasal cavity, typically in the form of a metered nasal spray, the drug and absorption enhancer deposits on the mucosal membrane surface inside the nose in the form of a thin layer. As a result, the drug and the absorption enhancer remain in close proximity at the mucosal membrane through which the drug is intended to be absorbed. Drug absorption enhancers have been used successfully to administer water soluble drugs in aqueous solution via oral gavage into fasted rodents.

However, in larger animals, owing to the large stomach volume relative to the when mixtures of drug absorption-enhancing agent are admitted into the stomach for oral administration, absorption enhancer and drug do not necessarily maintain relative proximity due to, for example, interaction with or dissolution into the stomach contents, thus limiting the effectiveness of the absorption enhancer and reducing drug absorption into systemic circulation. Therefore, innovative compositions are needed to enhance bioavailability of orally administered therapeutics, especially those that are poorly-water soluble.

### SUMMARY OF THE INVENTION

The present invention provides compositions having a non-aqueous matrix for enhancing bioavailability of orally administered therapeutics. The non-aqueous matrix is immiscible with water but can dissolve both a therapeutic and absorption enhancer. This allows for them to be maintained in close proximity until contact is made with the gastrointestinal mucosa upon oral administration. Thus, an enhanced means to deliver the therapeutic and absorption enhancer while maintaining them in close proximity at the mucosal surface is proved.

Accordingly, in one aspect, the present invention provides a composition for oral delivery of a therapeutic agent. The composition includes: a) a non-aqueous matrix comprising an alkylsaccharide absorption enhancer; and b) at least one therapeutic agent soluble in the non-aqueous matrix.

In another aspect, the present invention provides a method of increasing the bioavailability of a therapeutic agent administered orally to a subject. The method includes orally administering to the subject a composition having: a) a non-aqueous matrix including an alkylsaccharide absorption enhancer; and b) at least one therapeutic agent soluble in the non-aqueous matrix, thereby increasing the bioavailability of the analog in the subject.

### BRIEF DESCRIPTION OF THE DRAWINGS

FIG. 1 is graphical representation of a plot illustrating an octreotide acetate uptake profile following subcutaneous delivery in sodium acetate buffer. The plot depicts serum concentrations of octreotide acetate five, 10, 15, 30, 60, 120 and 180 minutes after subcutaneous delivery of 30 mcg in sodium acetate buffer to male Swiss Webster mice (n=3 mice per time point). Each value represents mean±SEM octreotide acetate concentration. Error bars are contained within each point and ranged between 0.01 and 0.10 ng/ml.

FIG. 2 is a graphical representation of a plot illustrating an octreotide acetate uptake profile following oral delivery by gavage in non-aqueous matrix. The plot depicts serum concentrations of octreotide acetate five, 10, 15, 30, 60, 120 and 180 minutes after oral delivery (by gavage) of 30 mcg in 0.5% dodecyl maltoside (DDM) in a non-aqueous solution comprising 70% vitamin D, 20% benzyl alcohol, 10% absolute ethanol to male Swiss Webster mice (n=3 mice per time point). Each value represents mean octreotide acetate concentration.

FIG. 3 is a graphical representation of a plot illustrating a sumatriptan uptake profile following oral delivery by gavage in non-aqueous matrix in a canine model. The plot depicts serum concentrations of sumatriptan in a canine at zero through 180 min. following oral delivery (by gavage) of 25 mg sumatriptan in 1.0% DDM in a non-aqueous solution comprising cocoa butter. The solid circles represent plasma sumatriptan concentrations for doses not containing DDM (the control) and the solid squares show plasma sumatriptan concentrations for the sumatriptan doses containing DDM.

### DETAILED DESCRIPTION OF THE INVENTION

The present invention is based on innovative compositions which allow for increasing bioavailability of orally administered therapeutic agents. The compositions include a non-aqueous matrix having an alkylsaccharide absorption enhancer, into which a therapeutic agent is dissolved. The non-aqueous matrix allows for the absorption enhancer and therapeutic to be maintained in a close proximity with each other until contact is made with mucosal surfaces following oral administration. Thus, bioavailability of the orally administered therapeutic is enhanced by the compositions described herein.

Before the present compositions and methods are described, it is to be understood that this invention is not limited to the particular composition, method, and experimental conditions described, as such composition, method, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the device” or “the method” includes one or more devices and methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In one aspect, the present invention provides a composition for oral delivery of a therapeutic agent. The composition generally includes a non-aqueous matrix including an alkylsaccharide absorption enhancer, the matrix being coformulated with at least one therapeutic agent which is soluble in the non-aqueous matrix. The non-aqueous nature of this invention is particularly advantageous for orally delivering water-sensitive and/or poorly water soluble therapeutic agents at a high dose.

In embodiments of the invention, a non-aqueous matrix is composed of one or more non-aqueous solvents. For example, the non-aqueous matrix may be composed of one or more of vitamin E, a tocopherol, a tocotrienol, a pharmaceutically acceptable oil or derivative thereof, an alcohol, a glycol, or mixtures thereof. It has been surprisingly found that the non-aqueous matrix described herein, particularly a non-aqueous matrix including a mixture of one or more of vitamin E, a tocopherol, a tocotrienol, a pharmaceutically acceptable oil or derivative thereof, an alcohol, and a glycol, and further including an alkylsaccharide absorption enhancer, enables stable solutions to be prepared containing high concentrations of therapeutic agents, and which can be successfully delivered orally.

Non-aqueous solvents for use in the present compositions include, by way of illustration, tocopherol and tocotrienol compounds including alpha-tocopherol, beta-tocopherol, gamma-tocopherol, delta-tocopherol, alpha-tocotrienol, beta-tocotrienol, gamma-tocotrienol, delta-tocotrienol tocophersolan, any isomers thereof, any esters thereof, any analogues, or derivatives thereof, and any combinations thereof. Additional solvents include synthetic tocopherols, vitamin E, and vitamin E TPGS (vitamin E polyethylene glycol succinate), as well as pharmaceutically acceptable oils including known vegetable or plant oils, such as, almond oil, hazelnut oil, walnut oil, peanut oil, poppyseed oil, olive oil, soybean oil, wheat germ oil, corn oil, sunflower, safflower oil, castor oil, and other vegetable or plant-based oils. Additional non-aqueous solvents include solid fats, such as cocoa butter which exists in a liquid oil state at or above about 37 degrees C., as well as derivatized plant oils, such as Cremaphor, and any mixtures or combinations thereof.

As used herein, vitamin E is used to refer to a group of fat-soluble compounds including tocopherols and tocotrienols. There are many different forms of vitamin E, of which gamma-tocopherol is the most common in the North American diet. Gamma-tocopherol can be found in plant oils such as corn oil, and soybean oil. Alpha-tocopherol, the most biologically active form of vitamin E, is the second most common form of vitamin E in the North American diet. This variant of vitamin E can be found most abundantly in wheat germ oil, sunflower, and safflower oils.

The non-aqueous matrix of the invention may further include additional non-aqueous solvents including either or both of an alcohol or a glycol. Examples of such alcohols suitable for inclusion in such mixtures include ethanol, propyl alcohol, butyl alcohol, pentanol, benzyl alcohol, any isomers thereof, or any combinations thereof. Glycols may include, by way of example, ethylene glycol, propylene glycol, glycerin, propylene carbonate, glycerol, glycofurol, polyethylene glycol, propylene glycol fatty acid esters, or any combinations thereof.

In an embodiment, the invention provides for the use of high concentrations of vitamin E, a tocopherol, a tocotrienol, and/or a pharmaceutically acceptable oil as a non-aqueous solvent component of the non-aqueous matrix. By high concentration, it is meant that the vitamin E, tocopherol, tocotrienol, and/or oil content of the matrix in which the therapeutic agent is dissolved is from about 50 to 100% by volume, 60 to 100% by volume, 65 to 100% by volume, 70 to 100% by volume, 75 to 100% by volume, or even 80 to 100% by volume. The remainder of the matrix may comprise other non-aqueous solvents (alone or in combination, such as an alcohol or glycol), and additionally at least one alkylsaccharide absorption enhancer. For example, the remainder of the matrix may include from about 1 to 30% by volume, 5 to 30%, 5 to 25% by volume, 10 to 20% by volume, or 15 to 30% by volume of one or more alcohols, glycols, or mixtures thereof. In one embodiment, matrix includes 75 to 95% by volume vitamin E, a pharmaceutically acceptable oil, or mixture thereof, and 5 to 25% by volume of an alcohol, such as ethanol or benzyl alcohol, alone or in combination.

As discussed above, examples of non-aqueous solvents that may be used in combination with vitamin E, a tocopherol, a tocotrienol, or a pharmaceutically acceptable oil include, but are not limited to, alcohols and glycols, such as ethanol, propyl alcohol, butyl alcohol, pentanol, benzyl alcohol, any isomers thereof, or any combinations thereof, ethylene glycol, propylene glycol, glycerin, propylene carbonate, glycerol, glycofurol, polyethylene glycol, propylene glycol fatty acid esters, or any combinations thereof. These solvents may be used alone or in mixture together with the vitamin E, tocopherol, tocotrienol, and/or oil to compose the non-aqueous matrix of the invention. Such a matrix comprises from 0 to 60%, preferably from 5 to 55%, from 5 to 50% by volume, 5 to 40% by volume, or 5 to 30% by volume of each non-aqueous solvent that is not vitamin E or oil, provided that the total amount of non-vitamin E or non-oil solvent does not exceed 60%, preferably 55% or 50% of the total volume of the matrix. The matrix may consist essentially of or consist of the vitamin E and/or pharmaceutically acceptable oil and optionally one or more of these non-aqueous solvents.

There are a number of different methods by which the compositions described herein can be produced. For example, in one method the non-aqueous matrix is first prepared by mixing together the matrix components along with the alkylsaccharide absorption enhancer in the required quantities by volume or by weight. The required amount of therapeutic agent and any other ingredients such as stabilizers may then be weighed into a suitable vessel, a portion of the matrix added (e.g. 90% of final amount) and the mixture stirred until the agent is dissolved. The solution is then made up to the required weight or volume by adding more of the therapeutic agent to the non-aqueous matrix. In another method, the therapeutic agent (and any other ingredients if appropriate) is weighed into a suitable vessel and the exact weight of each solvent and alkylsaccharide added. The mixture is then stirred until the therapeutic agent is dissolved. Any of these methods may be modified by a heating step to expedite or enhance incorporation of the therapeutic agent into the non-aqueous matrix. For example, the matrix may be heated to about or above 37 degrees C., such as to 37 to 50 degrees C., or higher. Further, following any of these methods, the final drug solution may be filtered if necessary.

As discussed herein, the composition of the invention further includes an alkylsaccharide absorption enhancer, which when combined with the therapeutic agent containing non-aqueous matrix of the invention, the bioavailability of the agent is increased upon oral administration. As used herein, “alkylsaccharide” refers to any sugar joined by a linkage to any hydrophobic alkyl, as is known in the art. The alkylsaccharide is nonionic as well as nontoxic and considered Generally Recognized As Safe, for food applications, sometimes referred to as a GRAS substance. Alkylsaccharides are available from a number of commercial sources and may be natural or synthesized by known procedures, such as chemically or enzymatically. An absorption enhancer considered to be orally compatible is one which does not cause severe or irreversible damage to gastrointestinal tissues.

Alkylsaccharides of the invention can be synthesized by known procedures, i.e., chemically, as described, e.g., in Rosevear et al., Biochemistry 19:4108-4115 (1980) or Koeltzow and Urfer, J. Am. Oil Chem. Soc., 61:1651-1655 (1984), U.S. Pat. No. 3,219,656 and U.S. Pat. No. 3,839,318 or enzymatically, as described, e.g., in Li et al., J. Biol. Chem., 266:10723-10726 (1991) or Gopalan et al., J. Biol. Chem. 267:9629-9638 (1992).

In various aspects, alkylsaccharides of the present invention may include, but are not limited to: alkylglycosides, such as octyl-, nonyl-, decyl-, undecyl-, dodecyl-, tridecyl-, tetradecyl-, pentadecyl-, hexadecyl-, heptadecyl-, and octadecyl- α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers.

As described above, the hydrophobic alkyl can thus be chosen of any desired size, depending on the hydrophobicity desired and the hydrophilicity of the saccharide moiety. For example, one preferred range of alkyl chains is from about 10 to about 24 carbon atoms. An even more preferred range is from about 10 to about 16 or about 14 carbon atoms. Similarly, some preferred glycosides include maltose, sucrose, and glucose linked by glycosidic linkage to an alkyl chain of 9, 10, 12, 13, 14, 16, 18, 20, 22, or 24 carbon atoms, for example, nonyl-, decyl-, dodecyl-, tridecyl, and tetradecyl sucroside, glucoside, maltoside, and the like. These compositions are nontoxic, since they are degraded to an alcohol or fatty acid and an oligosaccharide, and amphipathic. Additionally, the linkage between the hydrophobic alkyl group and the hydrophilic saccharide can include, among other possibilities, a glycosidic, thioglycosidic, amide, ureide, or ester linkage.

As used herein, a “saccharide” is inclusive of monosaccharides, oligosaccharides or polysaccharides in straight chain or ring forms, or a combination thereof to form a saccharide chain. Oligosaccharides are saccharides having two or more monosaccharide residues. The saccharide can be chosen, for example, from any currently commercially available saccharide species or can be synthesized. Some examples of the many possible saccharides to use include glucose, maltose, maltotriose, maltotetraose, sucrose and trehalose. Preferable saccharides include maltose, sucrose and glucose.

The alkylsaccharide of the invention can likewise consist of a sucrose ester. As used herein, “sucrose esters” are sucrose esters of fatty acids. Sucrose esters can take many forms because of the eight hydroxyl groups in sucrose available for reaction and the many fatty acid groups, from acetate on up to larger, more bulky fatty acids that can be reacted with sucrose. This flexibility means that many products and functionalities can be tailored, based on the fatty acid moiety used. Sucrose esters have food and non-food uses, especially as surfactants and emulsifiers, with growing applications in pharmaceuticals, cosmetics, detergents and food additives. They are biodegradable, non-toxic and mild to the skin.

In sugar chemistry, an anomer is either of a pair of cyclic stereoisomers (designated α or β) of a sugar or glycoside, differing only in configuration at the hemiacetal (or hemiketal) carbon, also called the anomeric carbon or reducing carbon. If the structure is analogous to one with the hydroxyl group on the anomeric carbon in the axial position of glucose, then the sugar is an alpha anomer. If, however, that hydroxyl is equatorial, the sugar is a beta anomer. For example, dodecyl β-D-maltoside and dodecyl α-D-maltoside are two cyclic forms of dodecyl maltoside and are anomers. The two different anomers are two distinct chemical structures, and thus have different physical and chemical properties. In one embodiment of the invention, the alkylsaccharide for use with the present invention is a β anomer. In an exemplary aspect, the alkylsaccharide for use in the invention is a β anomer of dodecyl maltoside, tridecyl maltoside or tetradecyl maltoside.

In an embodiment of the present invention, the alkylsaccharide used is a substantially pure alkylsaccharide. As used herein a “substantially pure” alkylsaccharide refers to one anomeric form of the alkylsaccharide (either the α or β anomeric forms) with less than about 2% of the other anomeric form, preferably less than about 1.5% of the other anomeric form, and more preferably less than about 1% of the other anomeric form. In one aspect, a substantially pure alkylsaccharide contains greater than 98% of either the α or β anomer. In another aspect, a substantially pure alkylsaccharide contains greater than 99% of either the α or β anomer. In another aspect, a substantially pure alkylsaccharide contains greater than 99.5% of either the α or β anomer. In another aspect, a substantially pure alkylsaccharide contains greater than 99.9% of either the α or β anomer.

In embodiments of the invention, orally compatible absorption enhancers intended for use in the invention are soluble in a the non-aqueous matrix and may include dodecyl maltoside, tetradecyl maltoside, tridecyl maltoside, decyl maltoside, undecyl maltoside, sucrose mono- or di-dodecanoate or mixtures thereof, sucrose mono- or di-tridecanoate or mixtures thereof, sucrose mono- or di-tetradecanoate or mixtures thereof, sucrose laurate, sucrose myristate, sucrose palmitate and sucrose cocoate which is a mixture of sucrose esters of varying chain lengths from 6 carbons to 18 carbons, with the predominant species in the mixture being sucrose dodecanoate and sucrose tetradecanoate which together comprise about 60% of the total mixture of chain lengths within the sucrose cocoate, all of which substances are considered GRAS substances for inclusion in or on foods by the FDA or US EPA.

The alkylsaccharide of the composition of the invention may be present at a level of from about 0.01% to 20% by weight. More preferred levels of incorporation are from about 0.01% to 5% by weight, from about 0.01% to 2% by weight, or from about 0.01% to 1%. In some embodiments the alkylsaccharide is present at a concentration between about 0.01% and 10% (w/v), about 0.05% and 20% (w/v), about 0.1% and 10% (w/v), or about 0.1% and 5% (w/v).

In addition to alkylsaccharides, a number of molecules have been screened for their ability to enhance transmucosal absorption and which may be incorporated into the non-aqueous matrix of the invention. Examples include, but are not limited to, aprotinin, benzalkonium chloride, cetylpyridinium chloride, chitosan, chitosan-4-thiobutylamidine, cyclodextrin, dextran sulfate, dodecyl azacycloheptyl-2-ketone, lauric acid, lysophosphatidylcholine, menthol, methoxysalicylate, methyloleate, phosphatidyl choline, polycarbophilm cysteinem poly-1-arginine, polyoxyethylene, polyoxyethylene-9-lauryl ether, polyoxyethylene-23-lauryl ether, polysorbate 80, EDTA, deoxycholate, glycocholate, glycodeoxycholate, lauryl sulfate, salicylate, taurocholate, taurodeoxycholate, taurodihydrofusidate, cyclopentadecalonide, and sodium N-8-[2-(hydroxybenzoyl)amino]caprylate (SNAC). In listing the anionic forms of various enhancers, it is understood that these may include the corresponding pharmaceutically acceptable salts (e.g., formed in combination with proton ion, sodium ion, potassium ion, lithium ion, calcium ion, magnesium ion, among others). In preferred embodiments, the EDTA is used along with one or more alkylsaccharides.

The non-aqueous matrices described herein are suitable for producing compositions for oral delivery of a wide range of therapeutic compounds. One skilled in the art would appreciate that it will be a straightforward matter to determine whether a particular non-aqueous matrix is suitable for use in combination with a particular drug on the basis of the teaching in this application. For example, this can be done by measuring the solubility of the agent in the matrix. The solubility can be tested by adding an excess of the agent to the vehicle and stirring the mixture for 24 hours at room temperature. Undissolved drug is then removed by filtration or centrifugation and the solution is assayed for dissolved drug content by an appropriate analytical method, such as high performance liquid chromatography.

While the non-aqueous matrix of the invention may include compounds of varying solubility, even those which are water soluble by incorporating surfactants, detergents or the like, the non-aqueous matrix is particularly suitable for use with agents which have a solubility in water at 20 degrees C. of not more than about 1 mg/ml. Such drugs are often referred to in the literature as “very slightly soluble” (solubility in water at 20 degrees C. of from 0.1 to 1 mg/ml) and “practically insoluble” or “insoluble” (for both, solubility in water at 20 degrees C. of less than 0.1 mg/ml).

A definition of high water solubility is provided in FDA guidance to Industry. Specifically the FDA states a drug substance is considered highly soluble when the highest dose strength is soluble in <250 ml water over a pH range of 1 to 7.5. In terms of the present invention, solubility of drugs in a non-aqueous matrix is what is most relevant. Solubility should be sufficient to allow an effective dose of drug to be dissolved in a small volume of non-aqueous matrix capable of being encapsulated ideally in one or two gelatin capsule for dosing at a single administration. Dosing multiple times per day allows a greater amount of drug to be administered if solubility is limiting or if the pharmacokinetic profile requires it. While a dose comprising one or two capsules is ideal for patient convenience, notwithstanding, for serious or life threatening diseases, a larger number of capsules of a drug may be acceptable. Typical volumes that may be encapsulated in a gelatin capsule range from less than 1 mL up to 2 mL or greater volumes. One mL capsules are among the most commonly used. Gel encapsulation services in hardshell gel or soft gel capsules are offered by multiple vendors such as Catalent, Somerset, N.J. or Fusion Formulations, Tempe, Ariz.

Some therapeutic agents (drug compounds) suitable for use in this invention include, but are not limited to, antibiotics and antimicrobial agents, such as tetracycline hydrochloride, leucomycin, penicillin, penicillin derivatives, erythromycin, sulphathiazole and nitrofurazone; antimigraine compounds, such as naratriptan, sumatriptan, zolmitriptan, rizatriptan, eletriptan, frovatriptan, aInitidan, avitriptan, almotriptan or other 5-HT1 agonists; vasoconstrictors, such as phenylephedrine hydrochloride, tetrahydrozoline hydrochloride, naphazoline nitrate, oxymetazoline hydrochloride and tramazoline hydrochloride; cardiotonics, such as digitalis and digoxin; vasodilators, such as nitroglycerin and papaverine hydrochloride; bone metabolism controlling agents, such as vitamin D and active vitamin D3; sex hormones; hypotensives; anti-tumour agents; steroidal anti-inflammatory agents, such as hydrocortisone, prednisone, fluticasone, prednisolone, triamcinolone, triamcinolone acetonide, dexamethasone, betamethasone, beclomethasone and beclomethasone dipropionate; non-steroidal anti-inflammatory drugs, such as acetaminophen, aspirin, aminopyrine, phenylbutazone, mefenamic acid, ibuprofen, diclofenac sodium, aceclofenac, piroxicam, meloxicam, tenoxicam, ketoprofen, dexketoprofen, flurbiprofen, ibuprofen, indomethacin, colchicines and probenecid; enzymatic anti-inflammatory agents, such as chymotrypsin and bromelain seratiopeptidase; anti-histaminic agents, such as diphenhydramine hydrochloride, chlorpheniramine maleate and clemastine; anti-tussive expectorants, such as codeine phosphate and isoproterenol hydrochloride; analgesics such as opioids (like diamorphine, hydromorphone, buprenorphine, fentanyl, oxycodone, codeine, morphine and its polar metabolites, such as morphine-6-glucuronides and morphine-3-sulphate), or combinations of opioids and other analgesic agents such as non-steroidal anti-inflammatory drugs; anti-emetics, such as metoclopramide, ondansetron, granisetron, tropisetron, palonosetron, dolasetron, dronabinol and nabilone; drugs for treatment of sleeping disorders, such as melatonin, zolpidem, zaleplon and zopiclone; drugs for treatment of asthma, such as salbutamol; drugs for treatment of erectile dysfunction such as apomorphine, sildenafil, tadalafil, vardenafil and alprostadil; antipsychotic drugs such as haloperidol, olanzapine, risperidone, ziprasidone, clozapine, loxapine, pimozide, zotepine, quetiapine, flupentixol, zuclopenthixol and sertindole.

A further class of compounds for use in the present invention is the benzodiazepines. These lipophilic drugs act on the central nervous system to cause sedation, hypnosis, decreased anxiety, muscle relaxation, anterograde amnesia and anticonvulsant actions and are widely used in medicine. Conditions which they can be used to treat include anxiety, epilepsy, insomnia, alcohol dependence, muscular disorders and mania. These drugs can also be used in premedication procedures and in veterinary practice. Examples of benzodiazepine drugs include, but are not limited to, alprazolam, chlordiazepoxide, clonazepam, clorazepate, diazepam, estazolam, flurazepam, halazepam, lorazepam, midazolam, nitrazepam, oxazepam, prazepam, quazepam, temazapem, bromazepam, flunitrazepam and triazolam, bentazepam, brotizolam, clotiazepam, delorazepam, ethyl loflazepate, etizolam, fludiazepam, ketozolam, loprazolam, lormetazepam, nordazepam, mexazolam, nimetazepam, pinazepam, tetrazepam and pharmaceutically acceptable salts thereof.

Additional examples of drugs which may be formulated in accord with the present invention include atorvastatin, fluticasone, salmeterol, clopidogrel, esomeprazole, amlodipine, etanercept, olanzapine, valsartan, risperidone, venlafaxine, pantoprazole, quetiapine, lansoprazole, losartan, alendronate, rosiglitazone, pioglitazone, simvastatin, glatiramer, rabeprazole, filgrastim, escitalopram, imatinib, zolpidem, donepezil, cetirizine, irbesartan, docetaxel, oxaliplatin, sertraline, oseltamivir, rosuvastatin, celecoxib, topiramate, ezetimibe, simvastatin, ezetimibe, bupropion, aripiprazole, lamotrigine, metoprolol, candesartan, tiotropium, sildenafil, telmisartan, risedronate, leuprolide, fenofibrate, ondansetron, valaciclovir, levofloxacin, anastrozole, tacrolimus, mycophenolate mofetil, latanoprost, carvedilol, gemcitabine, omeprazole, olmesartan, amlodipine, benazepril, duloxetine, sumatriptan, valproate, fentanyl, budesonide, zoledronate, ramipril, fluticasone, filgrastim, bicalutamide, pravastatin, tenofovir, emtricitabine, budesonide, formoterol, raloxifene, tamsulosin, pregabalin, paroxetine, lopinavir, tolterodine, tamsulosin, amoxiclav, estrogen, progesterone, goserelin, imiglucerase, levofloxacin, drospirenone, terbinafine, lamivudine, zidovudine, piperacillin, tadalafil, levetiracetam, mometasone, atazanavir, methylphenidate, ciclosporin, irinotecan, fexofenadine, amphetamine, ipratropium, salbutamol, nifedipine, moxifloxacin, meloxicam, clarithromycin, pravastatin, sevoflurane, efavirenz, linezolid, capecitabine, ziprasidone, ciprofloxacin, modafinil, fluvastatin, desloratadine, letrozole, oxcarbazepine, bosentan, sitaxsentan, imipenem, cilastatin, temozolomide, dorzolamide, diclofenac, tenofovir, pramipexole, memantine, ramipril, exenatide, erlotinib, azithromycin, cefdinir, finasteride, pemetrexed, meropenem, teriparatide, atomoxetine, fentanyl, fluticasone, glimepiride, lidocaine, eszopiclone, ibandronate, paclitaxel, tegaserod, sevelamer, levalbuterol, orlistat, enalapril, salmeterol, doxazosin, levothyroxine, famotidine, caspofungin, rivastigmine, voriconazole, amlodipine, niacin, gabapentin, abacavir, zidovudine, ropinirole, voglibose, vardenafil, metformin, bisoprolol, abacavir, lamivudine, alfuzosin, fluconazole, thalidomide, ranitidine, loratadine, phenylephrine, aspirin, naproxen, chlorpheniramine, dextromethorphan, leuprolide, octreotide, afpep, lanreotide, exendin-4, liraglutide, lixisenatide, taspoglutide, symlin, d-leu-ob-3, nafarelin, desmopressin, apomorphine, prochlorperazine alprazolam loxapine, diphenhydramine, ganirelix, tizanidine, buserelin, triptorelin, midazolam, naloxone, oxytocin, carbetocin, selegiline, diazepam, lysofylline, theophylline, testosterone, estradiol, estrogen, levonorgestrel, ethinyl estradiol, acetaminophen, ibuprofen, ketoprofen, sitaxsentan, bosentan, dextromethorphan, phenylephrine, pseudephedrine, hydrocodone, naproxen, guaifenesin, esomeprazole, lansoprazole, omeprazole, pantoprazole, rabeprazole, timoprazole, paroxetine, atomoxetine, duloxetine, fluoxetine, venlafaxine, metoprolol, propranolol, zolpidem, azithromycin, clarithromycin, erythromycin, rimonabant, tadalafil, lovastatin, pravastatin, simvastatin, alfuzosin, doxazosin, prazosin, terazosin, almotriptan, eletriptan, naratriptan, sumatriptan, zolmitriptan, losartan, linezolid, terbinafine, maraviroc, udenafil,nevirapine, pristiq, venlafaxine, bosentan, agomelatine ranolazine pirfenidone, oxybutynin, salmeterol, alfentanil, fentanyl, sufentanil, avosentan, laquinimod glipizide, vandetanib, aripiprazole, quetiapine, rolofylline, bupropion, carisoprodol, thiothixene, theophylline, theobromine, paraxanthine, tropisetron, palonosetron, prochlorperazine, amisulpride dapagliflozin, alosetron, lobeline, anastrozole, betaxolol, chlorcyclizine, chlorpromazine, granisetron, lopinavir bifeprunox, ondansetron 2promethazine risperidone acamprosate ciclesonide, carnitine, acetyl 1-carnitine, lumiracoxib, repaglinide, dimebolin, nicotine, cotinine, norcotinine, saxagliptin, aliskiren,dorzolamide, glyburide,meloxicam, memantine, nateglinide,orphenadrine, ropinirole, mifepristone, azelastine, casopitant,altretamine, aminocaproic acid, gefitinib, sibutramine, valproic acid, donepezil, cinacalcet, loperamide, etoposide,teniposide,edaravone, trimetrexate, tiotropium bromide, dronabinol, paliperidone, rolipram, olopatadine, pemirolast, tamsulosin, verapamil, gallopamil, nabilone, clozapine, remogliflozin, clemizole, solabegron, olanzapine, and cizolirtine, and deuterated forms of any of the above drugs in which one or more hydrogens is replaced by a deuterium atom.

Poorly water-soluble drugs are frequently compounded with solid excipients including absorption-enhancing surfactants and compressed into tablets or administered in capsules. Following administration, upon disintegration in the stomach, the surfactants, which by their amphiphilic nature are soluble in water, begin to dissolve whereas the poorly water-soluble drugs remain in a substantially insolubilized state. As a result, the absorption enhancers can diffuse away from the solid drug particles and when no longer in proximity to the drug particles can exert only reduced or no absorption enhancing effect. The presence of food substances within the stomach further confounds drug absorption by interaction with the surfactant.

However, as discussed herein, poorly water-soluble or amphiphilic molecules, including peptidic and non-peptides, can be solubilized and used in the non-aqueous matrix of the present invention. The structural class of peptide molecules found to be substantially orally absorbed using the compositions of the present invention includes both linear and cyclic peptides and non-peptide drugs. Generally, peptides comprised of about 100, 90, 80, 70, 60, 50, or 40 amino acids or less, containing either natural or non-natural amino acids, provide higher oral bioavailability than larger peptides. However, peptides drugs of any length may be utilized.

In the present description, the term non-natural amino acid is intended to mean amino acids other than the 20 naturally occurring L-amino acids generally accepted in the biological sciences to be common to most proteins, namely, alanine, cysteine, aspartic acid, glutamic acid, phenylalanine, lysine, histidine, isoleucine, lysine, leucine, methionine, asparagine, proline, glutamine, arginine, serine, threonine, valine, tryptophan, and tyrosine. For purposes herein, unnatural amino acids can include amino acids containing the D-isomer configuration since most proteins are comprised primarily or entirely of amino acids in the L-isomer configuration, notwithstanding the fact that D-amino acids do occur naturally in certain situations, including, for example, bacterial, fungal, and plant metabolism and byproducts. Examples of non-natural amino acids include, but are not limited to, D-amino acids, hydroxyproline, tert-leucine, hydroxyvaline, allothreonine, beta-dialkylserine, cyclohexylalanine, allylglycine, napthylalanine, pyridylalanine, 4-hydroxymphenylglycine, phenylglycine, homoserine, 3,4,dihydroxyphenylalanine, 4-chlorophenylalanine.

Peptides containing D amino acids and substituted side chains are generally accepted to exhibit improved stability in the gastrointestinal tract as a result of reduced proteolysis. Thus peptides for use with the present invention may be modified to include at least one non-natural amino acid. One skilled in the art would understand that a non-natural amino acid may be incorporated by a variety of methods known in the art, such as by addition, or alternatively by substitution or modification of an existing amino acid. As such, a peptide of the invention may include at least 10%, 20%, 30%, 40%, 50%, 60%, 70%, 80% or 90% of natural or L-amino acids, with the remainder being non-natural. For example, the peptide may include at least 90%, 91%, 92%, 93%, 94%, 95%, 96%, 97%, 98% or 99% natural amino acids.

As used herein, a cyclized peptide refers to a peptide that is generally cyclic in structure as a result of a linkage between two amino acids. Further, the terms “cyclic” and “cyclized” are used synonymously and refer to a peptide that has been synthetically cyclized or naturally occurs as a cyclic protein.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-natural amino acid. Additionally, such “polypeptides,” “peptides” and “proteins” include amino acid chains of any length, including full length proteins, wherein the amino acid residues are linked by covalent peptide bonds.

Cyclic peptides for use with the present invention may be readily synthesized by any known conventional procedure for the formation of a peptide linkage between amino acids. Such conventional procedures include, for example, any solution phase procedure permitting a condensation between the free alpha amino group of an amino acid residue having its carboxyl group or other reactive groups protected and the free primary carboxyl group of another amino acid residue having its amino group or other reactive groups protected.

The process for synthesizing the cyclic peptides may be carried out by a procedure whereby each amino acid in the desired sequence is added one at a time in succession to another amino acid residue or by a procedure whereby peptide fragments with the desired amino acid sequence are first synthesized conventionally and then condensed to provide the desired peptide. The resulting peptide is then cyclized to yield a cyclic peptide of the invention. A cyclic peptide can be obtained by inducing the formation of a covalent bond between an amino group at the N-terminus of the peptide, if provided, and a carboxyl group at the C-terminus, if provided. A cyclic peptide can also be obtained by forming a covalent bond between a terminal reactive group and a reactive amino acid side chain moiety, or between two reactive amino acid side chain moieties. One skilled in the art would know that the means by which a given peptide is made cyclic is determined by the reactive groups present in the peptide and the desired characteristic of the peptide.

Cyclic peptides for use with the present invention may be of a particular structural class which includes small to intermediate length cyclic peptides. Such peptides when orally administered via the composition described herein dramatically increases bioavailability. Cyclic peptides for use with the present invention may include from 2 to 50 amino acids, for example 3, 4, 5, 6, 7, 8, 9, 10, 11, 12, 13, 14 or 15 up to 50 amino acids, including 3, 4, 5, 6, or 7 up to 10, 15, 20, 25, 30, 35, 40, 45 or 50 amino acids. In some embodiments the peptide includes 2 to 20 amino acids, for example 5 to 15 amino acids, 5 to 13 amino acids, 7 to 13 amino acids, or 8 to 12 amino acids. In some embodiments, the peptide includes less than 50, 45, 40, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6 or 5 amino acids.

Examples of cyclic peptide antibiotics useful in the present invention include, but are not limited to daptomycin, vancomycin, bacitracin, gramicidin, grandamycin, viomycin, capreomycin, microcin J25, bacteriocin AS-48, rhesus theta defensin-1 (RTD-1), streptogramins, and polymyxins, such as polymyxin B, E and M.

As generally known in the art, proteolysis can be reduced by addition of protease inhibitors such as aprotinin, soybean trypsin inhibitor, and the like. Examples of protease inhibitors include bestatin, amastatin, boroleucin, borovaline, aprotinin, pepstatin A, leupeptin hemisulfate EDTA, EGTA, aminocaproic acid, chymostatin, and alpha-1-antitrypsin, among others. However, not all protease inhibitors are completely or partially soluble in non-aqueous solvents of the present invention. In a preferred embodiment of the present inventions, protease inhibitors that are at least partially soluble in the non-aqueous solvent are selected.

Stabilization in the gastrointestinal tract can also be accomplished by addition of a pH modifier to the drug formulation. Such pH modifiers may raise or lower the pH of the drug formulation. Yet another way to increase stabilization of a peptide in the gastrointestinal tract involves enteric coating, encapsulation, or time release coatings that prevent exposure of the drug formulation to parts of the gastrointestinal tract which may provide a hostile environment or to ensure release in portions of the gastrointestinal tract where peptides may be more stable.

The non-aqueous matrix of the present invention may further include preservatives. Examples of preservatives that may be used in the compositions of the present invention, include, but are not limited to preservatives such as ethylene diamine tetraacetic acid (EDTA), sodium azide, p-hydroxybenzoate and its analogs, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, chlorobutanol and m-cresol.

The term “subject” or “patient” as used herein refers to any individual or patient to which a composition is administered. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, and the like, and primates (including monkeys, chimpanzees, orangutans and gorillas) are included within the definition of subject.

In various embodiments, the bioavailability of an agent is increased by at least 5%, 10%, 15%, 20%, 30%, 40%, 50%, 60%, 70%, 80%, 90%, 100%, 150%, 200%, 250%, 500%, 1000% or greater when administered orally via a composition of the present invention as compared to the agent administered in the absence thereof.

Non-aqueous compositions of the present invention have the particular benefit of being containable within gelatin capsules, since the absence of water in these compositions prevents softening or dissolution of the gelatin or other gelling material which materials are water soluble. Gelatin capsules are widely used in pharmaceutical products and may be of a variety of types including soft gelatin capsules and hard (also called hard shell) gelatin capsules. Other gelling agents may be used to form pharmaceutical capsules such as plant polysaccharides or their derivatives like carrageenans, chitosans, pectins, and modified forms of starch and cellulose. Other ingredients can be added to the gelling agent solution like plasticizers such as glycerin and/or sorbitol to decrease the capsule's hardness, coloring agents, preservatives, disintegrants, lubricants and surface treatment. In a preferred embodiment, the compositions are enclosed in a gelatin capsule for oral administration. Capsules useful for use in the present invention may alternatively be fashioned from gelling agents other than gelatin such as those cited above.

In embodiments of the present invention, formulations are prepared containing an alkylsaccharide selected from among the group comprising n-decyl maltoside, n-undecyl maltoside, n-dodecyl maltoside, n-tridecyl maltoside, n-tetradecyl maltoside, n-pentadecyl maltoside, n-hexadecyl maltoside, sucrose mono-dodecanoate, sucrose mono-tetradecanoate, sucrose cocoate, in concentrations ranging from about 0.05%, 0.1%, 0.25% 0.5%, 1.5%, 3%, 5.0%, 10%, 20%, 30%, 40% to 50% (w/v), in a non-aqueous matrix including a pharmaceutically acceptable oil selected from alpha-tocopherol, beta-tocopherol, gamma-tocopherol, delta-tocopherol, alpha-tocotrienol, beta-tocotrienol, gamma-tocotrienol, delta-tocotrienol tocophersolan, any isomers thereof, any esters thereof, any analogues, or derivatives thereof, and any combinations thereof, synthetic tocopherol, vitamin E or vitamin E TPGS (vitamin E polyethylene glycol succinate), soybean oil, wheat germ oil, corn oil, sunflower, safflower oil, castor oil, cocoa butter, other vegetable or plant-based oils, and derivatized plant oils such as Cremaphor, and any mixtures or combinations thereof; and optionally an alcohol and/or glycol selected from ethanol, benzyl alcohol, propyl alcohol, butyl alcohol, pentanol, benzyl alcohol, any isomers thereof, ethylene glycol, propylene glycol, glycerin, and polyethylene glycol or any combinations thereof; and optionally one or more additional excipients such as, but not limited to, EDTA.

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1

### Oral Deliver of Octreotide in Non-Aqueous Compostions to Rats

Octreotide is an effective option for the medical treatment of patients with acromegaly. Octreotide is a cyclized and 8-mer peptide with the following sequence that is both cyclized and contains non-natural amino acids. The amino acid sequence of is cyclo-D-Phe-Cys-Phe-D-Trp-Lys-Thr-Cys-Thr-OL (disulfide bridge cys2-cys7) (SEQ ID NO: 1).

Octreotide is a synthetic analogue of somatostatin, with similar effects but a prolonged duration of action. Octreotide is routinely given by subcutaneous (s.c.) injection. DDM is known to increase oral absorption of octreotide when administered by oral gavage in aqueous buffer, however, this is not the case as for administration in a non-aqueous liquid dosage form. Oral gavage, which is conducted through a tube inserted to the esophagus into the stomach, is an unacceptable mode of oral administration in most cases, especially for routine administration to humans. Administration in the form of a gelatin capsule is preferable, however, aqueous solutions cannot be contained in gelatin capsules since gelatin is soluble in water.

This example describes a comparison of the pharmacokinetics of a 30 microgram subcutaneous injection and oral delivery of 30 microgram octreotide acetate compositions comprising increasing concentrations (0.25% 0.5%, 1.5%, 3% and 5.0%) n-dodecyl-beta-D-maltoside (DDM) in a non-aqueous solution comprising 70% vitamin D, 20% benzyl alcohol, 10% absolute ethanol, and 0.1% EDTA delivered by oral gavage to male Swiss Webster mice using the procedure previously described by Maggio and Grasso (Regulatory Peptides 167 (2011) 233-238).

Six week-old male Swiss Webster mice weighing approximately 30 g were obtained from Taconic Farms (Germantown, N.Y., USA). The animals were housed three per cage in polycarbonate cages fitted with stainless steel wire lids and air filters, and supported on ventilated racks (Thoren Caging Systems, Hazelton, Pa., USA) in the Albany Medical College Animal Resources Facility. The mice were maintained at a constant temperature (24° C.) with lights on from 07:00 to 19:00 h, and allowed food and water ad libitum until used for uptake studies. Lyophilized octreotide acetate was obtained from BCN (Spain) and Polypeptide Laboratories (Torrance, Calif.) and DDM was supplied by Aegis Therapeutics (San Diego, Calif.). For subcutaneous (s.c.) delivery, octreotide acetate was dissolved at a concentration of 30 μg/100 μl in 10 mM sodium acetate buffer containing 0.1% EDTA (pH 4.5). For oral delivery, octreotide acetate was dissolved at a concentration of 30 μg/200 ul in a composition comprising 70% vitamin E (tocopherol), 20% benzyl alcohol, 10% absolute ethanol, and 0.1% EDTA and 0.5%, 1.5% or 3.0% DDM and administered by gavage. At time zero (0), octreotide acetate was delivered subcutaneously or by gavage to each mouse.

Following treatment, the mice were transferred to separate cages for the designated time period. Five, 10, 15, 30, 60, 120 or 180 min after octreotide acetate delivery, the mice (n=three per time point) are anesthetized with isoflurane (5%) and exsanguinated by cardiac puncture. Euthanasia was confirmed by cervical dislocation. The blood is collected in sterile nonheparinized plastic centrifuge tubes and allowed to stand at room temperature for 1 h. The clotted blood was rimmed from the walls of the tubes with sterile wooden applicator sticks. Individual serum samples were prepared by centrifugation for 30 min at 2600×g in an Eppendorf 5702R, A-4-38 rotor (Eppendorf North America, Westbury, N.Y., USA). The serum samples in each experimental group were pooled and stored frozen until assayed for octreotide acetate content by EIA. Octreotide acetate concentrations in the pooled serum samples were assayed in triplicate with a rat/mouse octreotide enzyme immunoassay assay (EIA) kit obtained from Peninsula Laboratories, LLC (San Carlos, Calif.) according to the instructions supplied by the manufacturer.

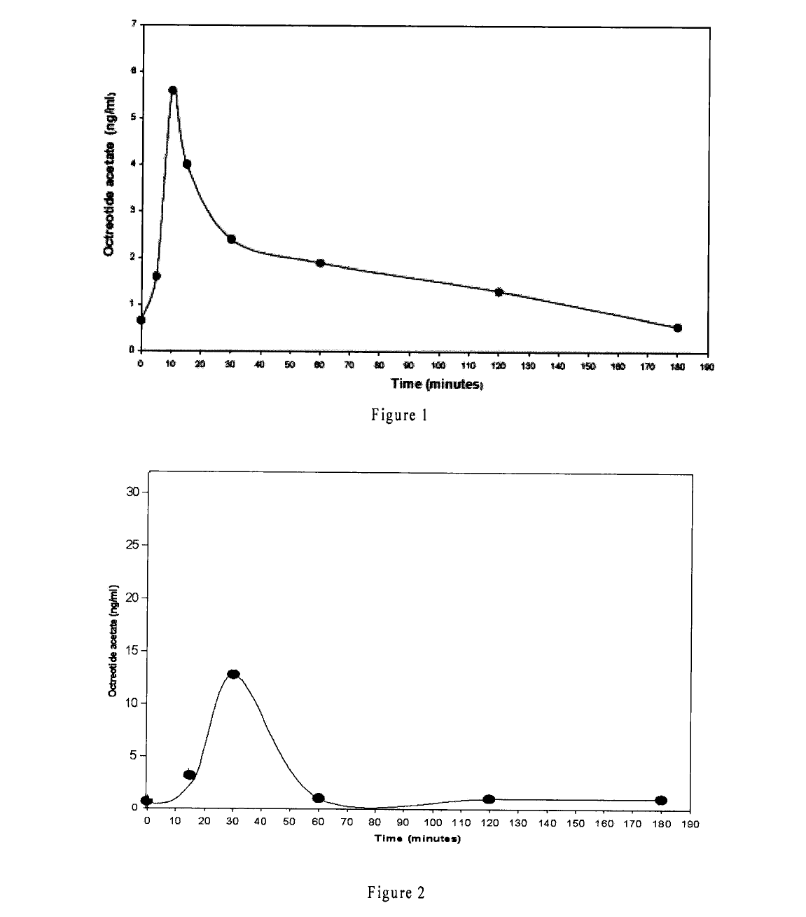

Serum concentrations of octreotide acetate vs. time following s.c. and oral delivery were plotted using the graphics program SigmaPlot 8.0 (SPSS Science, Chicago, Ill., USA). The area under each curve (AUC) is calculated with a function of this program. Examples of data obtained for s.c. injection and oral gavage are shown in FIGS. 1 and 2. The value obtained for s.c. injection is arbitrarily set at 1.0. Relative bioavailability is determined by comparing all other AUC values to 1.0 expressed as a ratio in Table 1 below.

TABLE 1

| | Relative Bioavailability |
|---|---|
| Octreotide Composition | (AUC(oral)/AUC s.c. injection) |
| 30 μg s.c. octreotide in acetate buffer | 1.0 |
| 30 μg octreotide in non-aqueous | 1.3 |
| formulation, 0.5% DDM | |

### Example 2

### Gelatin Encapsulated Vitamin E and Alcohol Non-Aqueous Matrix

A 400IU vitamin E softgel capsule (Mfgr. Nature Made) was evacuated using a 1 mL tuberculin syringe. The vitamin E (as alpha tocopheryl acetate) was compounded with absolute ethanol (Sigma-Aldrich) in the ratio of 85%:15%; and alternatively with benzyl (Sigma-Aldrich) alcohol and ethanol in the ratio of 80%:10%:10%. Approximately 1 mL of the liquid was injected through the top of the empty gelatin capsule while the capsule is held in a vertical position. A second perforation, also oriented at the top of the capsule, was made using the syringe allowing air to escape as the capsules were filled without causing the liquid to run out of the capsule. Both perforations were then sealed with a drop of warm water or warm gelatin solution (0.5 mg/mL) and allowed to set.

### Example 3

### Gelatin Encapsulated Soybean Oil/Alcohol Non-Aqueous Matrix

A 400IU vitamin E softgel capsule (Mfgr. Nature Made) was evacuated using a 1 mL tuberculin syringe. The vitamin E was discarded. Soybean oil was compounded with absolute ethanol (Sigma-Aldrich) in the ratio of 85%:15%; and alternatively with benzyl (Sigma-Aldrich) alcohol and ethanol in the ratio of 80%:10%:10%, then sealed with a drop of warm water or warm gelatin solution (0.5 mg/mL) and allowed to set.

### Example 4

### Gelatin Encapsulated Ibuprofen in Soybean Oil/Alcohol Non-Aqueous Matrix

A non-aqueous matrix was prepared as described in Example 3 and 200 mg of ibuprofen are dissolved per 0.75 mL volume of the non-aqueous liquid matrix. The liquid may be heated slightly to 50 degrees C. to facilitate dissolution. Using the procedure described in Example 3, approximately 0.75 mL liquid containing ibuprofen was injected through the top of the empty gelatin capsule while the capsule was held in a vertical position. A second perforation, also oriented at the top of the capsule, was made using the syringe allowing air to escape as the capsules were filled without causing the ibuprofen solution to run out of the capsule. Both perforations were then sealed with a drop of warm water or warm gelatin solution (0.5 mg/mL) and allowed to set.

### Example 5

### AFPep Solution in Vitamin E/Alcohol Non-Aquesous Matrix

AFPep is a first-in-class agent useful for treatment of breast cancer and other diseases. It is a 9-amino acid, cyclic peptide derivative of a natural human protein (α-fetoprotein, AFP). AFPep is active after oral administration, is well tolerated, and has a unique mechanism of action. AFPep is useful against breast cancer. Extensive research shows that AFPep stops the growth of human breast cancer growing in vitro or as xenografts in mice. In addition, AFPep prevents development of breast cancer in carcinogen-exposed animals. AFPep is useful against uterine fibroids, prostate cancer, and the glioblastoma form of brain cancer.

AFPep was compounded at a concentration of 2 mg/mL with alpha tocopheryl acetate and absolute ethanol (Sigma-Aldrich) in the ratio of 85%:15%; and alternatively with benzyl (Sigma-Aldrich) alcohol and ethanol in the ratio of 80%:10%:10%. Approximately 1 mL of the liquid was injected through the top of the empty gelatin capsule while the capsule was held in a vertical position. A second perforation, also oriented at the top of the capsule, made using the syringe allowing air to escape as the capsules are filled without causing the liquid to run out of the capsule. Both perforations were then sealed with a drop of warm water or warm gelatin solution (0.5 mg/mL) and allowed to set.

### Example 6

### Gelatin encapsulated AFTep in vitamin e/alcohol non-Aqueous Matrix

Approximately 1 mL of the AFPep solution of Example 5 was injected through the top of an empty gelatin capsule while the capsule is held in a vertical position. A second perforation, also oriented at the top of the capsule, was made using the syringe allowing air to escape as the capsules are filled without causing the liquid to run out of the capsule. Both perforations were then sealed with a drop of warm water or warm gelatin solution (0.5 mg/mL) and allowed to set.

### Example 7

### Gelatin Encapsulated Octreotide in Vitamin E/Alcohol Non-Aqueous Matrix

Approximately 1 mL of the octreotide solution of Example 1 was injected through the top of an empty gelatin capsule while the capsule was held in a vertical position. A second perforation, also oriented at the top of the capsule, was made using the syringe allowing air to escape as the capsules are filled without causing the liquid to run out of the capsule. Both perforations were then sealed with a drop of warm water or warm gelatin solution (0.5 mg/mL) and allowed to set.

### Example 8

### Oral Administration of Octreotide

Gelatin capsules containing 30 ug of octreotide in 70% vitamin E (tocopherol), 20% benzyl alcohol, 10% absolute ethanol, containing 0.5% DDM as described in Example 1 was placed in the mouth and swallowed. A small amount of water, typically less than 100 mL, may be taken at the same time to facilitate swallowing.

### Example 9

### Oral Administration of AFPep

Gelatin capsules containing 2 mg of AFPep in 70% vitamin E (tocopherol), 20% benzyl alcohol, 10% absolute ethanol, containing 1% DDM as described in example 1 was placed in the mouth and swallowed. A small amount of water, typically less than 100 mL, may be taken at the same time to facilitate swallowing.

### Example 10

### Oral Administration of Exendin-4

Gelatin capsules containing 30 micrograms of octreotide in 70% vitamin E (tocopherol), 20% benzyl alcohol, 10% absolute ethanol, containing 0.5% DDM as described in Example 1 was placed in the mouth and swallowed. A small amount of water, typically less than 100 mL, may be taken at the same time to facilitate swallowing.

### Example 11

### Oral Administration of Liraglutide

Gelatin capsules containing 3.6 mg of octreotide in 70% vitamin E (tocopherol), 20% benzyl alcohol, 10% absolute ethanol, containing 0.5% DDM as described in Example 1 was placed in the mouth and swallowed. A small amount of water, typically less than 100 mL, may be taken at the same time to facilitate swallowing.

### Example 12

### Compositions with Various Absorption Enhancers

Solutions were prepared according to the composition listed in the table below by dissolution in the specified non-aqueous matrices in 13 mm dia. glass test tubes. Solutions may be warmed gently at 37 degrees C. or 45 degrees C. to accelerate dissolution where desired. Test tubes are inspected visually for lack of opacity or presence of solid material. Clear liquid appearance indicates complete solution. All solutions were found to be stable upon storage at room temperature once dissolution is complete. For purposes of this example, commercially available Vitamin E gel caps may be manually emptied using a tuberculin syringe and subsequently refilled with the formulations where specified below and sealed with a drop of water or gelatin solution as described previously above. Commercially available hard shell gel caps may be filled manually and sealed by wetting the nested gelatin surface with water prior to joining the two halves of the gelatin capsule. A slight rotation may be employed upon joining the two halves to insure sealing of the wetted surfaces. Care should be taken to avoid contaminating the outside of the capsule surface that will be sealed by wetting with water since this may compromise the seal. Cocoa butter can be melted at slightly above 37 degrees C. to facilitate dissolution of drug.

TABLE 2

| | | | Absorption | |
|---|---|---|---|---|
| | | | enhancer | |
| | Alcohol/Glycol | Alcohol/Glycol | (0.1%-15% | |
| Solvent (fraction) | (fraction) | (fraction) | w/v) | Capsule type |
| Vit. E, Alpha-Tocopherol | Benzyl alcohol | Ethanol (10%) | Dodecyl | Soft gel |
| (Sigma Aldrich) (80%) | (10%) | | maltoside | |
| Soy bean oil (Sigma | Benzyl alcohol | Ethanol (15%) | Tetradecyl | Hard gel |
| Aldrich) (75%) | (10%) | | maltoside | |
| Corn oil (Sigma Aldrich) | Benzyl alcohol (5%) | Ethanol (5%) | Sucrose | — |
| (90%) | | | dodecanoate | |
| Safflower oil (Sigma | Benzyl alcohol | Ethanol (10%) | Tridecyl | Soft gel |
| Aldrich) (80%) | (10%) | | maltoside | |
| Vitamin E TPGS | Benzyl alcohol | — | Hexadecyl | Soft gel |
| (Cognis GmbH) (90%) | (10%) | | maltoside | |
| Vitamin E TPGS | — | Ethanol (15%) | Sucrose | Soft gel |
| (Cognis GmbH) (85%) | | | cocoate | |
| Cremophor EL (Sigma | Benzyl alcohol | Ethanol (10%) | Dodecyl | Soft gel |
| Aldrich) (80%) | (10%) | | maltoside | |
| Cocoa butter, NF | — | — | Dodecyl | — |
| (Spectrum Chemicals) | | | maltoside | |
| (100%) | | | | |

### Example 13

### Compositions with Various Drugs Soluble in Non-Aqueous Matrices

The following drugs, in the quantities specified, were dissolved in each of the compositions described in Example 10 above. Ibuprofen (200 mg), acetaminophen (325 mg), ketoprofen (75 mg), phenylephrine (10 mg), pseudephedrine (60 mg), dextromethorphan (15 mg), hydrocodone (5 mg), and naproxen (250 mg). The compositions were warmed at 37 degrees C. or 45 degrees C. to facilitate absorption. After 3 hours, solutions that appeared clear upon visual inspection were considered to be in complete solution and judged to be acceptable for therapeutic applications.

### Example 14

### Compositions with Sumatriptan in a Non-Aqueous Matrix

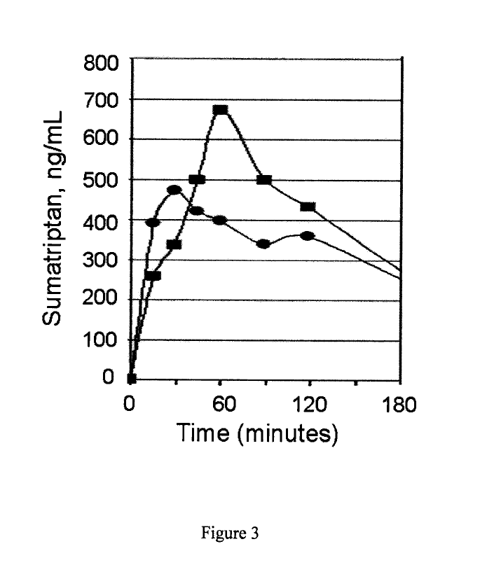

FIG. 3 shows the pharmacokinetic data for oral administration of sumatriptan in a dog at zero through 180 min. Sumatriptan, 2.5 g, was dissolved in 100 mL of cocoa butter at 45 deg. C. (in the liquid state) resulting in a solution having a sumatriptan concentration of 25 mg/mL. The solution was divided into two equal 50 mL portions. To one was added 500 mg DDM. Both solutions were kept at 45 degrees C. until the DDM is seen to have completely dissolved upon visual inspection yielding a 1% DDM solution in cocoa butter. The solutions were then cooled to about 4-10 degrees C. to allow the cocoa butter to solidify. One mL portions of the solidified cocoa butter-sumatriptan-DDM solution and one mL portions of the cocoa butter-sumatriptan (no DDM) control were formed into spherical balls and administered to beagle dogs by oral gavage. Blood samples were collected at timed intervals and plasma sumatriptan concentrations measured using a standard clinical assay. The results are shown in FIG. 3. The solid circles represent plasma sumatriptan concentrations for doses not containing DDM (the control) and the solid squares show plasma sumatriptan concentrations for the sumatriptan doses containing DDM, showing an approximate 19% increase in oral absorption of sumatriptan based upon the relative AUC's.

### Example 15

### Compositions with Diazepam in a Non-Aqueous Matrix

A solution containing 80% vitamin E and 20% dehydrated ethanol USP was prepared into which is dissolved 70 mg/mL of diazepam USP and 5 mg/mL, n-dodecyl maltoside. The solution was heated at 40 to 45° C. until the diazepam and dodecyl maltoside are fully dissolved at which point the solution is allowed to cool to room temperature (approx. 22-25° C.). Normal, healthy human test subjects were administered an amount of drug solution given orally, which provided a dose of approximately 35 mg of diazepam. Blood was collected immediately before administration and at selected time points after administration. Plasma blood levels of the drug were assayed for each blood samples. Pharmacokinetic curves showing blood plasma drug concentration versus time were constructed and the pharmacokinetic parameters are determined. Similar bioavailability to intravenous administration is observed with a Tmax of approximately 1.5 hours and a Cmax of approximately 270 ng/mL.

Although the invention has been described with reference to the above example, it will be understood that modifications and variations are encompassed within the spirit and scope of the invention. Accordingly, the invention is limited only by the following claims.

What is claimed is:

1. A composition for delivery of a therapeutic agent, the composition comprising:
a) a non-aqueous matrix comprising an alkylsaccharide absorption enhancer; and
b) at least one therapeutic agent soluble in the non-aqueous matrix.

2. The composition of claim 1, wherein the alkylsaccharide has an alkyl chain including between 10 to 16 carbons.

3. The composition of claim 1, wherein the alkylsaccharide is linked by glycosidic linkage to a maltose.

4. The composition of claim 1, wherein the alkylsaccharide is selected from the group consisting of: dodecyl maltoside, tridecyl maltoside, tetradecyl maltoside, sucrose dodecanoate, or sucrose cocoate.

5. The composition of claim 1, wherein the alkylsaccharide is a β-anomer.

6. The composition of claim 5, wherein the alkylsaccharide is tetradecyl-β-D-maltoside or dodecyl-β-D-maltoside.

7. The composition of claim 1, wherein the alkylsaccharide is present at a concentration between about 0.01% and 20% (w/v).

8. The composition of claim 7, wherein the alkylsaccharide is present at a concentration between about 0.01% and 10% (w/v), about 0.05% and 20% (w/v), about 0.1% and 10% (w/v), or about 0.1% and 5% (w/v).

9. The composition of claim 1, wherein the non-aqueous matrix comprises a non-aqueous solvent.

10. The composition of claim 9, wherein the non-aqueous matrix comprises a tocopherol, a tocotrienol, vitamin E, vitamin E TPGS, pharmaceutically acceptable oil, an alcohol, a glycol, or combination thereof.

11. The composition of claim 10, wherein the alcohol is ethanol, propyl alcohol, butyl alcohol, pentanol, benzyl alcohol, or combination thereof.

12. The composition of claim 10, wherein the glycol is ethylene glycol, propylene glycol, glycerin, propylene carbonate, glycerol, glycofurol, polyethylene glycol, propylene glycol fatty acid esters, or combination thereof.

13. The composition of claim 1, wherein the composition is formed by dissolving the therapeutic agent in the non-aqueous matrix.

14. The composition of claim 13, wherein the composition is heated at least about 37 degrees C.

15. The composition of claim 14, wherein the composition is heated to at least about 37 to 50 degrees C.

16. The composition of claim 1, wherein the composition is free of an aqueous solvent.

17. The composition of claim 1, wherein the composition is encapsulated in an erodable matrix.

18. The composition of claim 1, wherein the erodible matrix comprises gelatin.

19. A method of increasing the bioavailability of a therapeutic agent administered orally to a subject, comprising orally administering to the subject a composition, the composition comprising:
a) a non-aqueous matrix comprising an alkylsaccharide absorption enhancer; and
b) at least one therapeutic agent soluble in the non-aqueous matrix, thereby increasing the bioavailability of the analog in the subject.

20. The method of claim 19, wherein the alkylsaccharide has an alkyl chain including between 10 to 16 carbons.

21. The method of claim 19, wherein the alkylsaccharide is linked by glycosidic linkage to a maltose.

22. The method of claim 19, wherein the alkylsaccharide is selected from the group consisting of: dodecyl maltoside, tridecyl maltoside, tetradecyl maltoside, sucrose dodecanoate, or sucrose cocoate.

23. The method of claim 19, wherein the alkylsaccharide is a β-anomer.

24. The method of claim 23, wherein the alkylsaccharide is tetradecyl-β-D-maltoside or dodecyl-β-D-maltoside.

25. The method of claim 19, wherein the alkylsaccharide is present at a concentration between about 0.01% and 20% (w/v).

26. The method of claim 25, wherein the alkylsaccharide is present at a concentration between about 0.01% and 10% (w/v), about 0.05% and 20% (w/v), about 0.1% and 10% (w/v), or about 0.1% and 5% (w/v).

27. The method of claim 19, wherein the non-aqueous matrix comprises a non-aqueous solvent.

28. The composition of claim 27, wherein the non-aqueous matrix comprises a tocopherol, a tocotrienol, vitamin E, vitamin E TPGS, pharmaceutically acceptable oil, an alcohol, a glycol, or combination thereof.

29. The composition of claim 28, wherein the alcohol is ethanol, propyl alcohol, butyl alcohol, pentanol, benzyl alcohol, or combination thereof.

30. The composition of claim 28, wherein the glycol is ethylene glycol, propylene glycol, glycerin, propylene carbonate, glycerol, glycofurol, polyethylene glycol, propylene glycol fatty acid esters, or combination thereof.

31. The method of claim 19, wherein the composition is formed by dissolving the therapeutic agent in the non-aqueous matrix.

32. The method of claim 31, wherein the composition is heated at least about 37 degrees C.

33. The method of claim 32, wherein the composition is heated to at least about 37 to 50 degrees C.

34. The method of claim 19, wherein the composition is free of an aqueous solvent.

35. The method of claim 19, wherein the composition is encapsulated in an erodable matrix.

36. The method of claim 19, wherein the erodible matrix comprises gelatin.
